(19) United States
(12) Patent Application Publication
(10) Pub. No.: US 2004/0266726 A1
(43) Pub. Date: Dec. 30, 2004

(54) Anticancer compositions

(76) Inventor: Akikuni Yagita, Tokyo (JP)

Correspondence Address: KILYK & BOWERSOX, P.L.L.C., 53 A EAST LEE STREET, WARRENTON, VA, 20186, US

(21) Appl. No.: 10/494,813

(22) PCT Filed: Nov. 5, 2002
(86) PCT No.: PCT/JP02/11513

(30) Foreign Application Priority Data
Nov. 6, 2001 (JP) 2001341115
Feb. 18, 2002 (JP) 200240840

(51) Int. Cl. A61K 31/715; A61K 35/72

(52) U.S. Cl. 514/54; 424/195.16; 536/123.12

(57) ABSTRACT

To provide an anticancer composition. More specifically, the anticancer composition according to the present invention was successfully provided by newly finding the fact that a composition comprising a marine yeast-derived ingredient having β1,3 glucan structure is an unprecedentedly effective IL-12 inducer and further newly discovering that the composition can hold promise of NK and NKT cell activating capabilities as a result of oral administration of a marine yeast-derived ingredient having β1,3 glucan structure.

This application claims the benefit of priority to Japanese Patent Application Nos. 2001-341115 and 2002-040840, of which full contents are incorporated herein by reference.

### TECHNICAL FIELD

The present invention relates to provision of a new IL-12 inducer. More specifically, the present invention relates to IL-12 inducer comprising a marine yeast-derived ingredient having β1,3 glucan structure each. Further, the present invention relates to an anticancer composition that can hold promise of NK and NKT cell activating capabilities by oral administration of a marine yeast-derived ingredient having β1,3 glucan structure.

### BACKGROUND OF THE INVENTION

In selecting substances useful for prevention or treatment of cancer (malignant neoplasms), emphasis has hitherto been placed on their direct effect on cancer cells. While immunostimulators have been recognized as useful for cancer treatment, all compounds obtained as immunostimulators are feeble in their anticancer effect, leaving sufficient cancer treatment effect unattained both by immunotherapy alone and combination of immunotherapy and chemical therapy.

The present inventor, MD. Yagita, noting previously the usefulness of substances inducing interleukin 12 (IL-12) in vivo as an epoch-making method in cancer treatment, discovered that processed shiitake mycelium has that function, thus established a cancer treatment method that might be described as “novel immunotherapy for cancer” (NITC). IL-12 has been unusable as an anticancer drug despite its anticancer effect, because of the fact that patients are unable to endure treatment due to its side effects when IL-12 itself is directly administered in vivo. However, the preparation containing the processed shiitake mycelium reported by Yagita achieved outstanding curing- and life-prolonging effects in cancer treatment. That is, Yagita achieved the purpose of cancer treatment by administering the processed shiitake mycelium in effective amounts sufficient to induce IL-12 in vivo (Japanese Patent Application Laid-Open Publication No. 1998-139670).

The IL-12 has activating and augmenting effects on killer T cell through the route of TNFα−>IFNγ−>IL-12−>CTL activation. That is, augmentation of IL-12 production holds promise of anticancer effect by activating and augmenting killer T cell.

Yagita also reported, aside from the system of IL-12 production augmentation, that NKT cell activation is useful for anticancer effect. Taniguchi et al discovered a specific glicolipid antigen recognized by a specific T cell antigen receptor (TCR), Vα24Vβ11, carried in NKT cell, and reported that this antigen is a galactosylceramide. Further, they proved that, in a cancer-bearing mouse administered with α galactosylceramide, NKT cell is activated and metastasis suppressed, although the cancer disappearance is not observed.

It is reported that there is NK cell antigen receptor (NKR-P1; natural killer receptor P1) as another receptor in NKT cell (Feature Article—Basics and Clinicals of NKT Cell: Saishin Igaku Vol. 55, No. 4, 2000, P.818-823). Yagita found that NKR-P1 also participates in NKT cell activation and that this activation enhances anticancer effect.

It is reported that NK cell also plays a part in anticancer effect in vivo. However, the facts were proved by Yagita that NK cell activation and clinical anticancer effect are not correlated and that the amount of IL-12 production induced and NK cell activation exhibit a completely inverse correlation. Therefore, the credibility of NK cell playing a part in anticancer effect in human has been questioned.

MD. Yagita has studied hitherto various in vivo IL-12 inducers, and found a new IL-12 inducer (ILY registered trademark: Orient Cancer Therapy Co., Ltd., trade name) derived from bracket fungus mycelium, taking cancer cycle into consideration. Discovering that mushroom mycelium containing β1,3 glucan has antitumor effect and that its antitumor property results from cytokine (IL-12) activating totally Thl immunity, MD. Yagita has applied for patents for new use of products such as trade names AHCC, ILX, ILY, Krestin and SPG.

### SUMMARY OF THE INVENTION

It is one object of the present invention to provide a further useful IL-12 inducer, and more particularly to provide a more effective IL-12 inducer even in the case of the cancer that has progressed to a serious stage (progressive cancer, terminal cancer) . It is another object of the present invention to provide an anticancer composition that can hold promise of NK and NKT cell activating capabilities by oral administration of a marine yeast-derived ingredient having β1,3 glucan structure.

The present invention found as a result of study on yeasts as new substances, that a composition comprising a marine yeast-derived ingredient having β1,3 glucan structure each, is an unprecedentedly effective IL-12 inducer and that such a composition is an anticancer composition that can hold promise of NK and NKT cell activating capabilities by oral administration of a marine yeast-derived ingredient having β1,3 glucan structure, thus successfully provided an anticancer composition according to the present invention.

That is, the present invention consists of:

1. An IL-12 inducer comprising a marine yeast-derived ingredient having β1,3 glucan structure.

2. The IL-12 inducer according to 1, wherein a marine yeast-derived ingredient having β1,3 glucan structure is ingested orally in vivo from 10 to 2000 mg/kg of body weight/day in amounts.

3. The IL-12 inducer according to 1 or 2, wherein the inducer is a health aid food preparations intended for oral ingestion.

4. A cancer treatment method, wherein a marine yeast-derived ingredient having β1,3 glucan structure is ingested with IL-12 inducing capability as treatment marker.

5. A cancer treatment method, wherein a marine yeast-derived ingredient having β1,3 glucan structure is ingested with NK cell activating capability and/or NKT cell activating capability as treatment marker.

6. A cancer treatment method, wherein a marine yeast-derived ingredient having β1,3 glucan structure is ingested with IL-12 inducing capability, NK cell activating capability, and/or NKT cell activating capability as treatment markers.

7. A method for screening a new controlling cancer drug with the IL-12 inducing capability as indicator, using a marine yeast-derived ingredient having β1,3 glucan structure as candidate compound.

8. The method for screening according to 7 with NK cell activating capability and/or NKT cell activating capability as indicator.

9. A controlling cancer drug using a marine yeast-derived ingredient having β1,3 glucan structure obtained by the screening method for screening according to 7 or 8.

10. A commercial medium using natural laws, on which information described in 4 to 9 is carried.

11. A commercial method utilizing the commercial medium according to 10.

### BRIEF DESCRIPTION OF THE DRAWINGS

FIG. 1 illustrates the effect on the tumor volume, with 6d indicating the results of the sixth day;

FIG. 1-2 illustrates the effect on the tumor volume, with 9d and 13d respectively indicating the results of the ninth and thirteenth days;

FIG. 2 illustrates the effect on the amount of IL-12 induced, with 7d indicating the results of the seventh day;

FIG. 2-2 illustrates the effect on the amount of IL-12 induced, with 10d and 14d respectively indicating the results of the tenth and 14^{th} days;

FIG. 3 illustrates clinical example 1;

FIG. 4 illustrates clinical example 2;

### DETAILED DESCRIPTION OF PREFERRED EMBODIMENT

A marine yeast-derived ingredient, the main ingredient of the present invention, is a yeast having β1,3 glucan structure. The yeast was studied using trade name Y-1095 (Sankyo Yeast M) derived from a marine yeast , Saccharomyces cerevisiae (Alpenrose use) and the like. As a result of study, it was discovered that a marine yeast-derived ingredient having β1,3 glucan structure is a powerful IL-12 inducer and more particularly so in progressive and terminal cancer. Further, it was newly discovered that such marine yeast-derived ingredient is an anticancer composition that can hold promise of NK and NKT cell activating capabilities by oral administration of a marine yeast-derived ingredient having β1,3 glucan structure.

The present inventor found that among IL-12 production inducers are not only substances such as AHCC that induce IL-12 production particularly effectively in early-stage cancer patients but also substances such as yeast-derived substances having β1,3 glucan structure according to the present invention that also deliver IL-12 production inducing effect characteristically on progressive and terminal cancer patients.

The composition or the health aid food preparations intended for oral administration of the present invention are effective for treatment against cancers such as lung cancer, lung adenoma, thymoma, thyroid cancer, bladder cancer, colon cancer, rectal cancer, caecum cancer, ureteric cancer, breast cancer, cervical cancer, brain tumor, tongue cancer, pharynx cancer, nostril cancer, larynx cancer, stomach cancer, liver cancer, bile duct cancer, testis cancer, ovary cancer, uterine body cancer, malignant melanoma and liposarcoma, but are not limited thereto. Particularly, they are preferably administered to those with low IL-12 value (e.g., 7.8 pg/ml or less) even when IL-12 production inducer such as AHCC (Aminoup) is administered.

The IL-12 production inducer, NK cell activator and NKT cell activator according to the present invention are employed in a prescription that can induce or enhance activation thereof and further maintain activation using results from immunity measurement method as indicators. That is, based on the indicators, the quantity administered and the period over which they are administered are selected for using them so as to induce or enhance activation thereof and further maintain activation. The IL-12 production inducer, NK cell activator and NKT cell activator are preferably orally ingested. Naturally, they can be parenterally ingested (including administration into vein or muscle) by reducing the quantity administered and preparing them into a quality that can endure parenteral ingestion.

The effect of administration of a marine yeast-derived ingredient having β1,3 glucan structure on immunity was measured and examined using the following markers:

(1) IL-12 Production Capability

Although CTL activation can be judged by CD8 (+) perforin production capability, there are two CD8(+) perforin values, namely, cytotoxic T lymphocyte (CTL) and suppressor T cell (STC) The former impairs cancer cell, whereas activation of the latter results in cancer multiplication. Therefore, evaluation cannot be conducted with their absolute values. However, a cell is judged as being CTL if IFNγ is 10 IU/ml or more or if the IL-12 value is 7.8 pg/ml or more and as STC if IFNγ and IL-12 are low. For this reason, it is possible to make evaluation using IFNγ production capability (IFNγ value) or IL-12 production capability (IL-12 value).

(2) NK and NKT Cell Activating Capabilities

Both NK and NKT cells carry NKR-P1 (NK cell receptor CD161 (+)). For the former, NK cell count can be measured by the CD3(−)CD161(+) surface marker, whereas its activation can be judged by the CD3(−)CD161(+) perforin production capability. For the latter, on the other hand, NKT cell count can be measured by the CD3(+)CD161(+) surface marker, whereas NKT cell activation can be measured by its perforin production capability.

In cancer treatment, therefore, it is possible to evaluate effector cell using the measurement items given below in novel immunotherapy for cancer (NITC) and common immunotherapies alike. More specifically, CTL activation can be evaluated by IFNγ or IL-12 inducing production capability. NK cell activation can be evaluated by CD3(−)CD161(+) or CD3(−)CD161(+) perforin value. NKT cell activation can be evaluated by CD3(+)CD161(+) or CD3(+)CD161(+) perforin value.

Measurement methods for cells and individual markers are illustrated below.

(NKT Cell Measurement) (NK Cell Measurement) (CD8 Measurement)

Measurement of NKR-P1-bearing NKT cell can be conducted by measuring cell surface antigens (CD3 and CD161) existing specifically on the NKT cell surface. More specifically, peripheral blood lymphocyte are examined in respect of cells with positive CD3 and positive CD161 (CD3(+)CD161(+)). That is, CD3 and CD161, NKT cell surface antigens, are measured by the two-color test using flow cytometry with monoclonal antibody. Here, the term “NKT cell activation” refers to the fact that the CD3(+)CD161(+) NKT cell proportion in lymphocyte is 10% or more and preferably 16% or more. The term “NKT cell activating capability” denotes a capability of increasing the NKT cell proportion to 10% or more and preferably 16% or more, or a capability of further augmenting the NKT cell proportion more than before administration of a certain substance.

Likewise, the term “(CD3(−)CD161(+))” refers to examination of cell for negative CD3 and positive CD161. It has been confirmed that this method is useful for NK cell measurement in the present invention.

Further, the term “CD8(+)” denotes examination of cell for positive CD8. It has been confirmed that this method is useful for CTL activation measurement in the present invention.

In examples, using cancer patients' blood, blood cell were discriminated as positive or negative for cell surface antigens such as CD3, CD161 and CD8, with each cell proportion measured, as normally done, by the two-color test using flow cytometry. At this time, each of monoclonal antibodies for CD3, CD161 and CD8 was manufactured by Coulter or Becton Dickinson.

(Perforin Production Cell Measurement)

Two among CD3, CD161 and CD8, cell surface antigens, and perforin are measured in respect of peripheral blood lymphocyte as normally done by the two-color test using flow cytometry. More specifically, sampled blood is added with fixative, thus fixing cells. After addition of membrane permeating solution, anti-perforin antibody (manufactured by Pharmingen) is added for reaction. Further, PRE-Cy5 labeled second antibody (manufactured by DAKO) is added for reaction, followed by addition of anti-CD3-PE (Coulter 6604627) antibody and anti-CD161-FITC (B-D) antibody for reaction, after which measurement is made by flow cytometry. Abbreviations in the figures are indicated as PERF.

(Sample Preparation for Cytokine Measurement)

First, mononuclear cell fraction is isolated from blood for preparation. Heparin-added peripheral blood is diluted twofold with phosphate buffered saline (PBS) and mixed, then the mixture is layered over Ficoll-Conray solution (specific gravity: 1.077), centrifuging at 400 G for 20 minutes, after which mononuclear cell fraction is collected. After washing, 10% fetal bovine serum (FBS)-added RPMI-1640 culture medium is added for preparation to provide a cell count of 1×10⁶. Phytohemagglutinin (manufactured by DIFCO) is added to 200 μl of the cell suspension thus obtained to provide a concentration of 20 μg/ml, and then the mixture is cultured using a 96 well micro plate under 5% CO₂ at 37° C. for 24 hours. The cell solution thus cultured is used as the Cytokine measurement sample.

(IL-12 Amount Measurement)

The amount of IL-12 induced can be measured using a measurement kit based on the enzyme-linked immuno sorbent assay (ELISA) without making indirect measurement as is done with human since a sufficient amount of IL-12 is induced in serum in the experimental examples using mice described below. In this system using mice, it is possible to examine for IL-12 production inducing capability by having them orally ingest IL-12 production inducing substance continuously and finding increase in amount of blood IL-12 thereafter.

It is to be noted that the amount of blood IL-12 is not directly measurable in humans due to existence of inhibitor in blood. For instance, measurement of the amount of IL-12 induced in a cancer patient is conducted using a cultured solution made available by first feeding a stimulant to peripheral blood mononuclear cell, isolated and prepared from the cancer patient's blood, culturing the mixture and centrifuging it for cell removal. The number of cells subjected to culture is 0.5×10⁶ cell/ml to 1×10⁷ cell/ml and preferably 1×10⁶ cell/ml. For cell-stimulating substance, meanwhile, phytohemagglutinin (PHA)—a conventionally used mitogen—is added to provide a final concentration of 0.1 to 100 μg/ml and preferably 1 to 20 μg/ml for culture. Cell-stimulating substance is not limited to PHA, and any substance may be used as long as the substance is capable of stimulating cells and thus causing them to produce immunobiological active substance in order to achieve the objects of the present invention. PMA (Phorbol 12-Myristate-13-Acetate), PMA+Ionomycin, LPS (Lipopolysaccharide) and PWM (Poke Weed Mitogen) are included in such substances. While IL-12 amount measurement itself may be performed using publicly known clinical or biochemical tests, a measurement kit available from R&D SYSTEMS or MBL is used that is based on the enzyme-linked immuno sorbent assay (ELISA). Here, the term “IL-12 production inducing capability” refers to a capability of augmenting the amount of IL-12, which is produced by peripheral blood mononuclear cell as a result of stimulation, to 7.8 pg/ml or more, or a capability of augmenting the amount of IL-12 produced more than before administration of a certain substance.

The composition intended for oral ingestion according to the present invention comprises, as an active ingredient having IL-12 production inducing capability, a marine yeast-derived ingredient having β1,3 glucan structure.

The composition intended for oral ingestion according to the present invention comprising a marine yeast-derived ingredient having β1,3 glucan structure for inducing IL-12 production differs considerably from AHCC that is publicly known for its IL-12 production inducing capability in individual stages of cancer progression.

The composition according to the present invention whose effective ingredient is a marine yeast-derived ingredient having β1,3 glucan structure exhibits a sufficient IL-12 production inducing capability in the initial stage of cancer and characteristically delivers an equivalent or more powerful IL-12 production inducing capability in progressed terminal cancer. On the other hand, while AHCC delivers a characteristic IL-12 production inducing capability in the initial stage of cancer, its inducing capability falls off as the cancer progresses.

The amount of administration of the composition intended for oral ingestion according to the present invention is 1 to 2000 mg/kg of body weight per day and preferably about 10 to 2000 mg/kg of body weight, with the composition preferably orally ingested one to several times per day over the time period of 10 days to one year. Naturally, the composition can be parenterally ingested by reducing the amount administered and preparing it into the quality such as that administration via parenteral ingestion can be endured.

A marine yeast-derived ingredient having β1,3 glucan structure, the main ingredient of the present invention is publicly known as food material. For instance, Sankyo Yeast M (a marine dry yeast) and the like are illustrated. It is to be noted that commercial products were used as samples in the present invention.

Oral preparations are prepared into tablets, powders, capsules, syrups, etc. Preparations can naturally be produced as such by mixing them with a necessary additive such as known filler, disintegrator, binder or lubricant through stereotyped means. Further, flavoring agent, colorant, perfume, stabilizer, disinfectant, antiseptic and the like may be added as necessary.

As described above, the present invention clarifies the relationship between the composition intended for oral ingestion comprising a marine yeast-derived ingredient having β1,3 glucan structure as effective ingredient, and the IL-12 production inducing capability during progression stages of cancer. Thus, a commercial medium carrying these pieces of information serves as means for differentiating values of the product. Therefore, the commercial medium carrying these pieces of information is extremely high in usefulness. Additionally, since these pieces of information, if used commercially, serve as means for differentiating the product values, a commercial method using these pieces of information is extremely high in usefulness.

Information such as that described above, if carried on a medium using natural laws, serves as a useful commercial medium, and the commercial medium provides a useful commercial method.

### EXAMPLE

While a detailed description will be given below of the present invention with reference to examples, the present invention is not limited thereto.

### Example 1

Isolation and use of a marine yeast (Saccharomyces cerevisiae)

Collection of isolation sample was carried out mainly at seashore of Pacific coast in Southern part of Tohoku district and in Kanto district, and 2061 marine water samples and 293 samples of seaweeds and marine small animals were collected. Isolation from marine water was carried out by filtering them through 0.45 μm mesh of membrane filter at the collection place, putting the filter on the agar medium for isolation, and culturing them in GasPak Anaerobic pot (manufactured by BBL, using hydrogen and carbon dioxide generator) for 10 days at 27° C. Also, isolation from seaweeds and marine small animals was carried out by putting about 1 g of sample into 9 ml of collection medium, culturing it under anaerobic condition, and subjecting small amount of the medium to streak culture on isolation agar medium. Culturing was carried out under anaerobic condition.

Identification of isolated strains was performed by studying morphological character and physiological character with the method of Van Der Walt and D. Yallow according to The Yeasts (ed. by N. W. K reger van Rij). Also, DNA-DNA homology test with S. cerevisiae standard strain was carried out, and they were determined as S. cerevisiae. As a result, 10 strains from marine water and 3 strains from seaweeds of S. cerevisiae were successively isolated.

Preparation of yeast (Saccaromyces cerevisiae) cells

(A) Sankyo marine dry yeast

(B) Alpenrose Saccaromyces cerevisiae

The shaking cultured cells in 500 ml of flask with 150 ml of YPD medium (10 g of Yeast extract, 20 g of polypepton, 20 g of glucose, 1000 ml of distilled water, pH 5.0) at 27° C. for 24 hr were used as seeding cells. 3 l of the above-described culture solution putted into jar fermentor for 5.0 l was sterilized (121° C., 40 minutes), and then the above-described shaking cultured cells was seeded and cultured at 25° C. for 48 hr. The cultured cell was subjected to centrifugation (8,000 rpm, 10 minutes), and separated into supernatant and precipitation. The obtained cells was washed twice with 0.85% saline, dried at 70° C. for 24 hr, and homogenized with a mortar to obtain powder of cell {(A): about 20 g, (B): about 22 g}.

### Example 2

Immunological antitumor effect and IL-12 production capability were examined using Saccharomyces cerevisiae (Alpenrose Saccharomyces cerevisiae (300 mg/kg)) and a marine dry yeast of Sankyo CO., Ltd. (Y-1095: trade name Sankyo Yeast M) (300 mg/kg), a marine fresh yeast of Sankyo CO., Ltd. (1 g/kg)) . It is to be noted that each was administered in adjusted amounts such that the β1,3 glucan content in all groups became identical to each other.

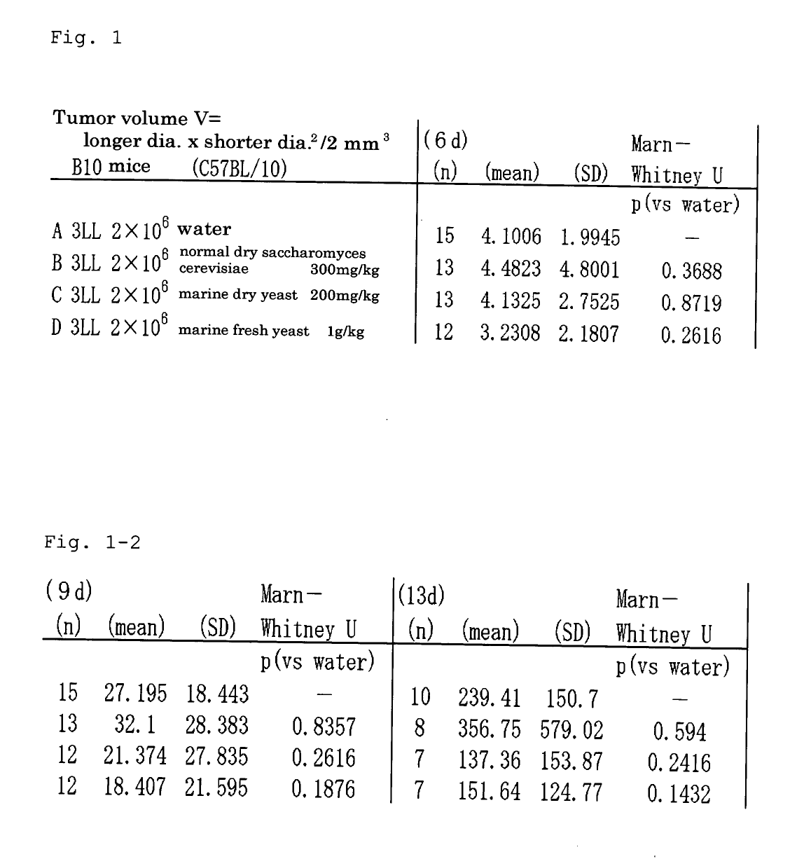

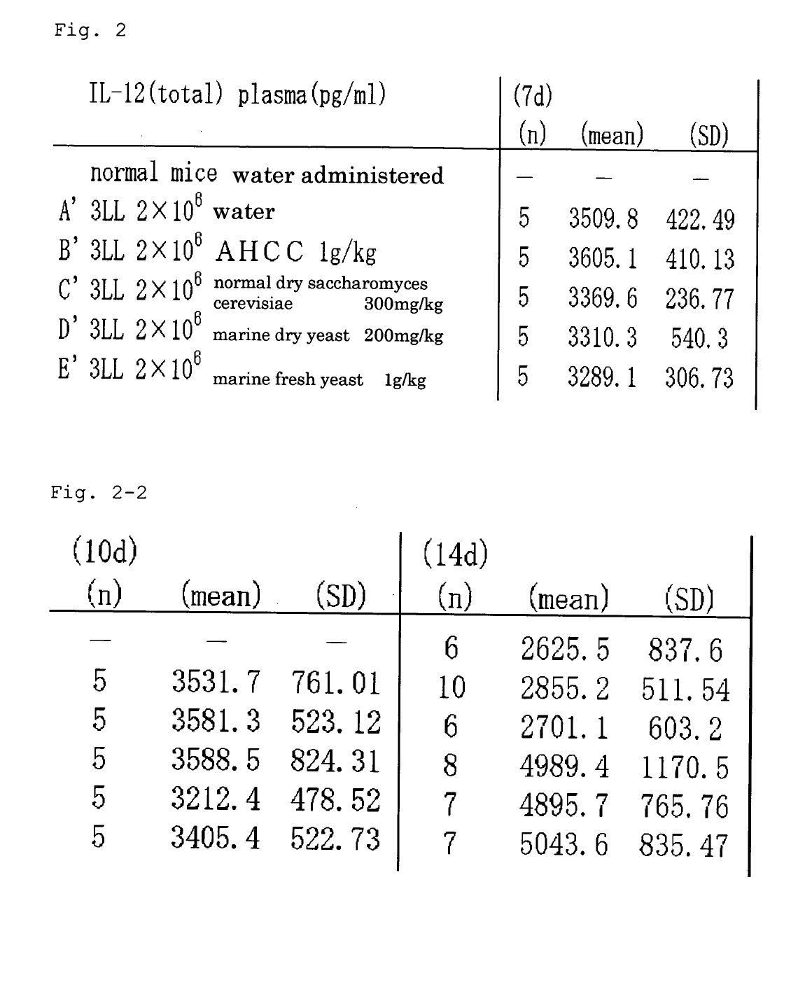

In the experiment, 3LL tumor were transplanted to B10 mice (C57BL/10), with a comparison made in terms of tumor volume on the 13^{th} day (FIGS. 1 and 1-2).

After the tumor transplantation, the tumor volume in control (A) for forced oral administration of water was 239.41±150 mm³ (the 13^{th} day ), whereas an increasing tendency was observed in the tumor volume for normal Saccharomyces cerevisiae/dry yeast (B) (300 mg/kg) as compared with the control.

On the other hand, an shrinking tendency was observed in the tumor volume for both a marine dry yeast of Sankyo CO., Ltd. (C, D) as compared with the control.

As for IL-12 concentration in blood, the group of a marine dry yeast of Sankyo CO., Ltd. showed a significant elevated value in IL-12 concentration relative to control (A) for forced oral administration of water (FIGS. 2, 2-2). IL-12 concentration was measured with Biotrak RPN2702 Interleukin-12total{(m)IL-12}, (p 40and p 70), mouse ELISA system kit from Amersham Pharmacia.

As compared with control (A′) for forced water oral administration, IL-12 showed an elevated value in all examination groups. However, as compared totally in respect of tumor shrinkage effect and IL-12 production comprehensively it was seemed that a marine dry yeast of Sankyo CO., Ltd. had the highest effect.

Further, although a marine dry yeast used in the present experiment is Y-1095, other marine dry yeast has the same effect (Table 1 as follows)

TABLE 1

| | Fermentation^{a} | | |
|---|---|---|---|
| Strain | Sucrose | Maltose | Salt tolerance^{b} |
| Marine isolate | | | |
| Y-990 | 3.49 | 3.19 | 8 |
| Y-995 | 3.47 | 3.31 | 8 |
| Y-997 | 3.48 | 3.27 | 8 |
| Y-1001 | 3.51 | 3.28 | 8 |
| Y-1002 | 3.52 | 3.20 | 8 |
| Y-1012 | 3.49 | 3.50 | 8 |
| Y-1095 | 3.53 | 3.37 | 8 |
| Y-1156 | 3.32 | 2.04 | 8 |
| Y-1140 | 3.43 | 2.52 | 8 |
| Y-1146 | 3.52 | 1.86 | 8 |
| Y-1160 | 3.42 | 2.17 | 8 |
| Y-1161 | 3.24 | 2.00 | 8 |
| Y-1164 | 3.44 | 2.47 | 8 |

^{a}CO₂ gas-producing activity (g)
^{b}Maximum NaCl concentration (%) for growth

### CLINICAL EXAMPLES

While a specific description will be given below of the present invention with reference to clinical examples, the present invention is not limited thereto. It is to be noted that the efficacy of therapies used was rated as Complete Remission (CR), Partial Remission (PR), No Change (NC) or Progressive Disease (PD) in accordance with the Standard for judgment of the efficacy of anticancer agent under GCP of the Japan Ministry of Health and Welfare.

### Clinical Example 1

M.Y. 59 y.o. Female Ovary Cancer

Administration of 6.0 g/day of ILX (registered trademarks), 3.0 g/day of ILY (registered trademark), 20 g/day of Better Shark LO and the like, basidiomycetes preparation, began on August 7, H.1X, first examined. This treatment method is named NITC by Yagita.

IL-12 production capability and NKT cell activity was enhanced and the tumor marker, CA15-3 (normal, not more than 30 U/ml) continued dropping from 100 U/ml, and CA125 (normal, not more than 35 U/ml) also continued dropping from 1200 U/ml. On July 1, H. 1#, all tumor marker as described above showed the value not more than normal value and the patient rated as CR.

However, into 17^{th} months after start of treatment, CA125 value began to increase, and CA72-4 and STN, tumor markers related to ovary cancer began to show abnormal value.

On September 14, H.1Y, 26^{th} months after start of treatment, oral administration of 6.0 g/day (divided by three, 2 g) of a marine yeast of SP-1 (trade name Y-1095 Sankyo Yeast M) started.

Into second months after the administration, CA125 dropped from 1900 U/ml to 120 U/ml, CA72-4 dropped from 38 U/ml to 3.0>U/ml, and into third months, all of CA125, STN antigen and CA72-4 showed the value not more than normal value.

During that period, by oral administration of 6.0 g/day (divided by three, 2 g) of SP-1, augmenting effect on IL-12 production capability showed from not more than 7.8 pg/ml to 16.1 pg/ml and 12.6 pg/ml.

From the above findings, it was verified that administration of a marine yeast-derived ingredient having β1,3 glucan structure is clinically effective. Additionally, correlation was observed between administration of a marine yeast-derived ingredient having β1,3 glucan structure, and augmentation of IL-12 production. As a result, it was confirmed that ingestion of a marine yeast-derived ingredient having β1,3 glucan structure is effective for cancer treatment using IL-12 inducing capability as treatment markers.

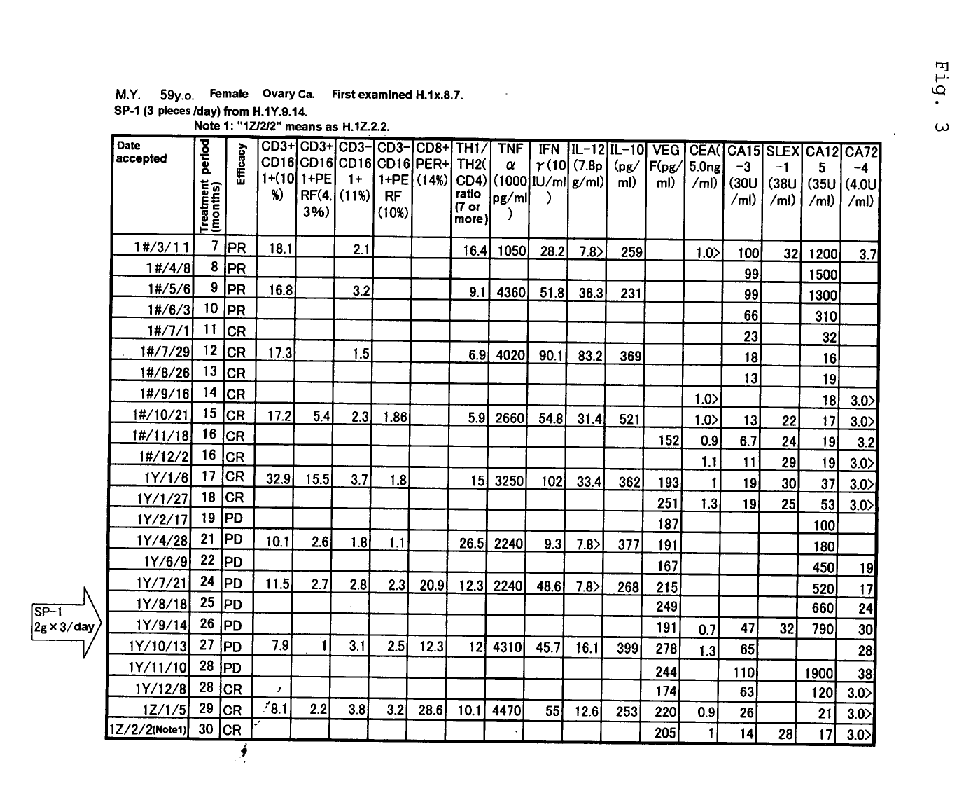

Detailed data is shown in FIG. 3.

### Clinical Example 2

M.K. 72 y.o. Male Multiple Cancer of Adenocarcinoma In Lung Cancer and Stomach Cancer

This is a case of multiple cancer of adenocarcinoma in the lung cancer and stomach cancer that visited the hospital due to be impossible to remove the cancer.

Oral administration of 6.0 g/day ILX (registered trademark), 3.0 g/day of ILY (registered trademark), 20 g/day of Better Shark LO began.

However, as for progression of cancer, tumor marker (CEA, NCC-ST439, CA15-3, SLX-1) not increased so that the patient rated as NC, and into 5^{th} months, SLX-1 became worse from 120 U/ml to 150 U/ml so that the patient rated as PD. Also, IL-12 production capability dropped.

For the reason, oral administration of 6.0 g/day (divided by three, 2 g) of SP-1 (trade name Y-1095 Sankyo Yeast M) started. Then, each tumor marker continued declining, IL-12 production capability recovered, and both of NK cell and NKT cell was activated, so that retained PR.

From the above findings, it was verified that administration of a marine yeast-derived ingredient having β1,3 glucan structure is clinically effective. Additionally, correlation was observed between administration of a marine yeast-derived ingredient having β1,3 glucan structure, and augmentation of IL-12 production, NK cell activating capability and/or NKT cell activating capability. As a result, it was confirmed that ingestion of a marine yeast-derived ingredient having β1,3 glucan structure is effective for cancer treatment using IL-12 inducing capability, NK cell activating capability and/or NKT cell activating capability as treatment markers.

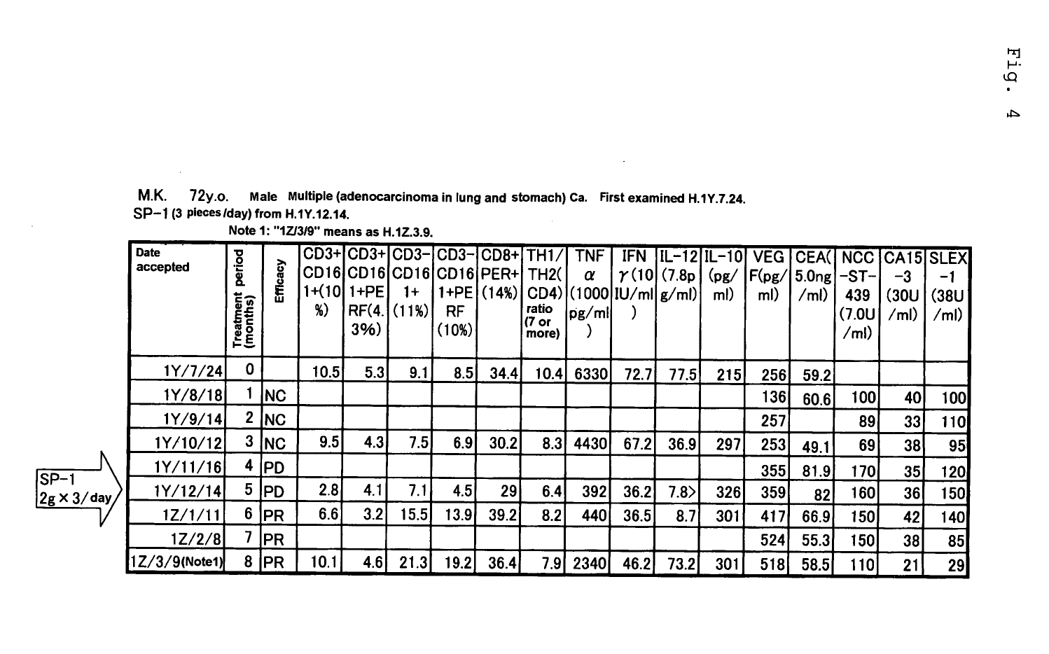

Detailed data is shown in FIG. 4.

Industrial Applicability

The anticancer composition according to the present invention was successfully provided by newly finding the fact that a composition comprising a marine yeast-derived ingredient having β1,3 glucan structure is an unprecedentedly effective IL-12 inducer and further discovering that the composition can hold promise of NK and NKT cell activating capabilities as a result of oral administration of a marine yeast-derived ingredient having β1,3 glucan structure.

1. An IL-12 inducer comprising a marine yeast-derived ingredient having β1,3 glucan structure.

2. (canceled)

3. (canceled)

4. A cancer treatment method, wherein a marine yeast-derived ingredient having β1,3 glucan structure is ingested with IL-12 inducing capability as treatment marker.

5. A cancer treatment method, wherein a marine yeast-derived ingredient having β1,3 glucan structure is ingested with NK cell activating capability and/or NKT cell activating capability as treatment marker.

6. A cancer treatment method, wherein a marine yeast-derived ingredient having β1,3 glucan structure is ingested with IL-12 inducing capability, NK cell activating capability, and/or NKT cell activating capability as treatment markers.

7. A method for screening a new controlling cancer drug with the IL-12 inducing capability as indicator, using a marine yeast-derived ingredient having β1,3 glucan structure as the candidate.

8. The method for screening according to claim 7 with NK cell activating capability and/or NKT cell activating capability as indicator.

9. A cancer controlling drug using a marine yeast-derived ingredient having β1,3 glucan structure obtained by the screening method according to claim 7.

10. A commercial medium utilizing a law of nature, on which information described in claim 4 is carried.

11. A commercial method utilizing the commercial medium according to claim 10.

12. An IL-12 inducer comprising a marine yeast-derived ingredient having β1,3 glucan structure, wherein the inducer is administered to progressive or terminal cancer patients.

13. The IL-12 inducer according to claim 12, wherein the progressive or terminal cancer:
1) ovary cancer,
2) adenocarcinoma in lung cancer, or
3) stomach cancer,

14. The IL-12 inducer according to claim 1, wherein a marine yeast-derived ingredient having β1,3 glucan structure is ingested orally in vivo from 10 to 2000 mg/kg of body weight/day in amounts.

15. The IL-12 inducer according to claim 1, wherein the inducer is the health aid food preparations intended for oral ingestion.

16. A cancer treatment method, comprising
measuring IL-12 inducing capability first,
administrating the IL-12 inducer of claim 1 to a patient, and
measuring again IL-12 inducing capability.
